(19) United States
(12) Patent Application Publication
(10) Pub. No.: US 2003/0083638 A1
(43) Pub. Date: May 1, 2003

(54) Disposable pants leg elastics

(76) Inventor: Kenneth John Molee, Suwanee, GA (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(21) Appl. No.: 09/985,168

(22) Filed: Nov. 1, 2001

(51) Int. Cl. A61F 13/15; A61F 13/20

(52) U.S. Cl. 604/385.27; 604/385.25

(57) ABSTRACT

The invention comprises an absorbent garment and a method for making an absorbent garment. An absorbent garment is provided comprising at least a pair of leg elastics attached to predetermined portions of at least one element of the garment to provide less or no cross core compression, and less bunching, gathering, or shirring of material in the crotch region of the garment. A method for making the absorbent garment also is disclosed.

### FIELD OF THE INVENTION

The present invention relates to absorbent garments. Specifically, the invention relates to an absorbent garment having elastic material extending laterally across the crotch region with substantially no tensile load.

### DESCRIPTION OF RELATED ART

Present-day all-in-one diapers are being replaced to an ever greater extent with pants-type diapers, or so-called training pants, for slightly older diaper-wearing children. Pants-type diapers have a number of desirable features. For example, pants-type diapers resemble conventional underpants and, thereby, can help somewhat older diaper-wearing children to learn to perform the operations required to put on conventional underpants as well as encourage the child to graduate to underpants. However, present pants-type garments can be difficult to put on, especially by young children. Furthermore, such garments are subject to loosening during use and may not fit smaller users very well.

Several efforts have been made to develop an absorbent garment/pants-type diaper with stretchable side panels. U.S. Pat. No. 5,843,068, the disclosure of which is incorporated by reference herein in its entirety, discloses a pants-type diaper comprising elastic side panels interconnecting front and back sections of a back sheet. The elastic side panels are a composite of dissimilar layers that are bonded together. The elastic side panels extend over a substantial distance along the lateral edges of the back sheet and have long bond seams joining the panels. Such a construction provides substantially more side coverage of a user than a belt, leading to less direct breathability. Furthermore, the panels' greater size and longer bond seams create smaller leg holes, which causes greater difficulty for a user attempting to put a foot through the leg hole; particularly for a younger user attempting to use the absorbent garment as a training pant without assistance. During application of the elastic elements, it is common for the elastics to extend laterally across the crotch region in tension and cause bunching and shirring in this area.

Various garments have been proposed whereby elastic elements are placed on or about the leg openings to provide a secure fit about the legs. Many garments also are equipped with standing leg gathers that are disposed inside the leg openings. These elastic elements are designed to provide a comfortable fit, while at the same time, retain body exudates.

U.S. Pat. Nos. 5,055,103, 5,147,487, 5,188,627 and RE 37,154 the disclosure of each of which is incorporated by reference herein in its entirety, disclose application of leg elastics to provide improved exudate containment.

### SUMMARY OF THE INVENTION

There exists a need to develop an absorbent garment having elastic elements disposed therein, whereby the garment provides an improved fit. There also exists a need to provide an absorbent garment having minimal or no cross core compression and bunching in the center of the crotch zone even though the garment is provided with elastic elements extending laterally across the crotch region. There also exists a need to provide a method of making an absorbent garment whereby the elastic elements that are provided across the crotch region of the garment are relatively close to one another.

It is therefore a feature of an embodiment of the invention to provide an absorbent garment having improved comfort and fit around the leg and crotch area. It is an additional feature of an embodiment of the invention to provide an absorbent garment having elastic elements that extend laterally across the crotch region, whereby there is minimal or no cross core compression or bunching in the center of the crotch region where the elastic elements are disposed. It is an additional feature of an embodiment of the invention to provide an absorbent garment whereby the elastic elements in the crotch region are relatively close to one another, and whereby little or no tension exists in or on the elastic elements in the crotch region.

In accordance with these and other features of various embodiments of the invention, there is provided an absorbent garment having a main body including a top sheet and a back sheet, the garment having a longitudinal dimension corresponding to the front and back of a wearer, and a lateral dimension corresponding to the left and right side of a wearer. The garment includes laterally extending front and rear waist edges, and a pair of left and right front and rear side edges, where at least the front waist edge and left and right front side edges form a front waist region, and at least the rear waist edge and left and right rear side edges form a rear waist region. The garment also includes left front and left rear arcuate edges joining the left front side edge to the left rear side edge, and right front and right rear arcuate edges joining the right front side edge to the right rear side edge. At least part of the arcuate edges form a crotch region that is disposed between the front and rear waist regions, and when the front and rear waist regions are brought together, the arcuate edges define leg openings.

The absorbent garment includes at least a pair of longitudinally opposing leg elastics that are disposed adjacent the arcuate edges, and that extend laterally across the crotch region of the garment The longitudinally opposing leg elastics are comprised of at least three segments, whereby the first segment is disposed adjacent one front or rear arcuate edge of the garment, the second segment extends laterally across the crotch region of the garment, and the third segment is disposed adjacent the laterally opposing front or rear arcuate edge of the garment. The leg elastics disposed in the longitudinal front portion of the garment do not contact the leg elastics disposed in the longitudinal rear portion of the garment. The first and third segments of the longitudinally opposing leg elastics are adhered to at least one other element of the garment under tension, and the second segment of the longitudinally opposing leg elastics is not adhered to another element of the garment. The leg elastics are applied to the garment in such a fashion that the second segment of the leg elastics are under no tensile load after application.

In accordance with another feature of an embodiment of the invention, there is provided a method of forming an absorbent garment comprising providing a back sheet material and a top sheet material to a forming station. The method also includes providing an absorbent core and at least one pair of leg elastics to the forming station and forming the garment by disposing the absorbent core and the at least one pair of leg elastics between the back sheet material and the top sheet material. The absorbent garment so formed then is cut to form a final absorbent garment having the following characteristics:

a longitudinal dimension corresponding to the front and back of a wearer, and a lateral dimension corresponding to the left and right side of a wearer, whereby the lateral dimension corresponds substantially to the machine direction of the forming station;

front and rear waist edges, and a pair of left and right side edges, where at least the front waist edge and left and right front side edges form a front waist region, and at least the rear waist edge and left and right rear side edges form a rear waist region;

left front and left rear arcuate edges joining the left front side edge to the left rear side edge, and right front and right rear arcuate edges joining the right front side edge to the right rear side edge, whereby at least part of the arcuate edges form a crotch region that is disposed between the front and rear waist regions, and when the front and rear waist regions are brought together, the arcuate edges define leg openings.

Providing the at least one pair of longitudinally opposing leg elastics comprises supplying at least one elastic element to the longitudinal front half of the garment, and at least one elastic element to the longitudinal rear half of the garment, whereby the elastic elements are supplied in a staggered fashion such that the elastic element supplied to one half of the garment is supplied shortly after the elastic element supplied to the other half of the garment.

Supplying the at least one elastic element to the longitudinal front or rear half of the garment comprises supplying an elastic element in a sinusoidal configuration so that the element includes three different segments, whereby the first segment is disposed adjacent the front or rear arcuate edge of the garment leading in the machine direction, the second segment extends laterally across the crotch region of the garment, and the third segment is disposed adjacent the laterally opposing front or rear arcuate edge of the garment trailing in the machine direction. The leg elastics disposed in the longitudinal front portion of the garment do not contact the leg elastics disposed in the longitudinal rear portion of the garment. The first and third segments of the longitudinally opposing leg elastics are adhered to at least one other element of the garment under tension, and the second segment of the longitudinally opposing leg elastic elements is applied in such a fashion that it is not adhered to another element, and such that it is under no tensile load after application.

These and other objects, features and advantages of various embodiments of this invention will become evident from the following description of the preferred embodiments of this invention, with reference to the accompanying drawings.

### BRIEF DESCRIPTION OF THE DRAWINGS

FIG. 1 depicts an embodiment of the present invention as it appears when worn by a user, with the back sheet partially cut away to show the absorbent core;

FIG. 2 is an exploded view of an embodiment of the present invention with elastic members shown in the elongated position for clarity, and the garment laid flat;

FIG. 3 depicts an embodiment illustrating the method by which the absorbent garment of the invention is made;

FIG. 4 illustrates the leg elastics of the garment of the invention in the relaxed position in the crotch region of the garment; and

FIG. 5 illustrates a preferred method of manufacturing the absorbent garment 10.

### DETAILED DESCRIPTION

As used herein, the terms “absorbent garment” and “garment” refer to items that absorb and contain fluid discharges and exudates, and more specifically refer to garments that are placed against or in proximity to the body of the user to absorb and contain various bodily discharges. A non-exhaustive list of examples of absorbent garments includes diapers, diaper cores, diaper covers, disposable diapers, training pants, feminine hygiene products and adult incontinence products. The claims are intended to cover all of the forgoing classes of absorbent garments, without limitation, whether disposable, unitary or otherwise. These classifications are used interchangeably throughout the specification, but are not intended to limit the claimed invention. The invention will be understood to encompass, without limitation, all classes of absorbent garments, including those described above.

Throughout this description, the term “adhered” denotes an association between the respective elements, and is not intended to limit the invention to adherence by means of an adhesive. The respective elements may be adhered to, or operatively associated with, one another by a variety of methods known in the art, including, but not limited to: using adhesives such as hot melt adhesives and construction adhesives, chemical or solvent bonding, ultrasonic welding, stitching, heat bonding, or any other method of affixation known or hereafter discovered. In addition, the expression “not adhered to another element” insofar as it relates to the second elastic element segment, denotes a situation where the second elastic element segment is not adhered to another element of the absorbent garment under tension. After applying the second elastic element segment, however, it may be adhered to, or operatively associated with, another element after it has assumed its relaxed position.

The invention is directed to an absorbent garment and to a method of making an absorbent garment. The absorbent garment preferably has at least a pair of longitudinally opposing elastic elements disposed on each side of the lateral center line of the absorbent garment. The elastic elements preferably are separated from one another at the crotch region, and are not adhered to another element of the garment under tension in a center portion of the crotch region. The elastic elements are not under any tension in the area where they are not adhered, and consequently, the elastic elements will not cause the absorbent garment to shirr, gather, or bunch in this region.

The absorbent garment of the invention preferably has a main body including a top sheet and a back sheet, a longitudinal dimension corresponding to the front and back of a wearer, and a lateral dimension corresponding to the left and right side of a wearer. The garment includes front and rear waist edges, and a pair of left and right front and rear side edges, where at least the front waist edge and left and right front side edges form a front waist region, and at least the rear waist edge and left and right rear side edges form a rear waist region. The garment also includes left front and rear arcuate edges joining the left front side edge to the left rear side edge, and right front and rear arcuate edges joining the right front side edge to the right rear side edge. At least part of the arcuate edges form a crotch region that is disposed between the front and rear waist regions, and when the front and rear waist regions are brought together, the arcuate edges define leg openings.

The absorbent garment includes at least a pair of longitudinally opposing leg elastics that are disposed adjacent the arcuate edges, and that extend laterally across the crotch region of the garment The longitudinally opposing leg elastics preferably are comprised of at least three segments.

The first segment is disposed adjacent one front or rear arcuate edge of the garment, the second segment extends laterally across the crotch region of the garment, and the third segment is disposed adjacent the other laterally opposing front or rear arcuate edge.

The leg elastics disposed in the longitudinal front portion of the garment do not contact the leg elastics disposed in the longitudinal rear portion of the garment. It is preferred that the leg elastics in the longitudinal front and rear portions of the garment are separated from one another by a distance of from about 0.1 mm to about 50 mm. The first and third elastic element segments of the longitudinally opposing leg elastics are adhered to at least one other element of the garment, preferably the top sheet or the back sheet under tension. The second elastic element segment of the longitudinally opposing leg elastics are not adhered to another element of the garment under tension. The leg elastics are applied to the garment in such a fashion that the second segment of the leg elastics is under no tensile load after application.

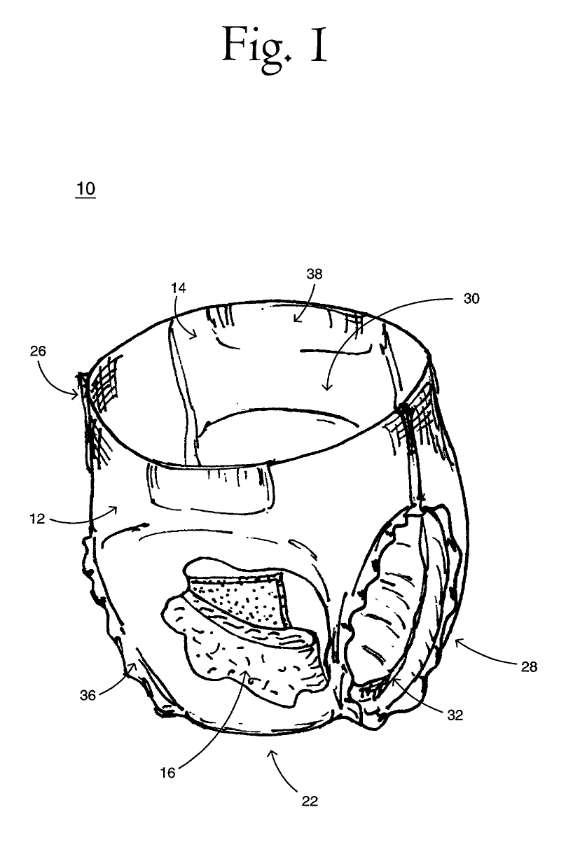

FIG. 1 depicts an embodiment of the present invention as it appears when worn by a user, with the main body partially cut away to show the absorbent core. In the depicted embodiment, the garment 10 is comprised of a main body having an exterior facing moisture impervious outer layer 12 or “back sheet,” and a moisture pervious body-contacting inner layer 14 or “top sheet.” An absorbent core 16 is disposed between the top sheet 14 and the back sheet 12. In the embodiment depicted in FIG. 1 the back sheet 12, top sheet 14, and core 16 comprise the main body of the garment, however in another embodiment of the invention the main body may be made from a separate sheet and the back sheet 12, top sheet 14, and core 16 may be assembled separately then attached to the main body.

In the embodiment of the present invention depicted in FIG. 1, the garment 10 further comprises various mechanisms for improving the fit of the garment 10 such as leg gathers 36 and standing leg gathers 32. Such gathers can be used to contract the leg holes 28 around the wearer's legs and body to prevent leakage. A garment 10 of the present invention may also comprise elastic or other fitting devices in the waist portions or other portions of the main body to help contain body exudates.

The various parts of the garment 10 are operatively associated with one another in such a manner that the garment will maintain its desired structure during use. The parts may be operatively associated with one another by a variety of methods known in the art, including, but not limited to: using adhesives such as hot melt adhesives and construction adhesives, chemical or solvent bonding, ultrasonic welding, stitching, heat bonding, or any other method of affixation known or hereafter discovered. All of the parts may be joined to each adjacent part, but some parts may not be joined to others. In one embodiment, the top sheet 14 and back sheet 12 are bonded to one another around their perimeter regions, thereby encasing and holding the absorbent core 16 in place without having to directly join the absorbent core 16 to any parts of the garment 10. The top sheet 14 or back sheet 12 may also be operatively associated with the absorbent core 16. As understood herein, the term “operatively associated” includes directly joining one part to another, indirectly joining parts together through one or more intermediary parts, whether those intermediary parts are described herein or not, joining parts in such a manner that unjoined parts are captured or held in their proper place, and any other suitable joining means that maintains the structural integrity of the garment 10 for the duration of its use.

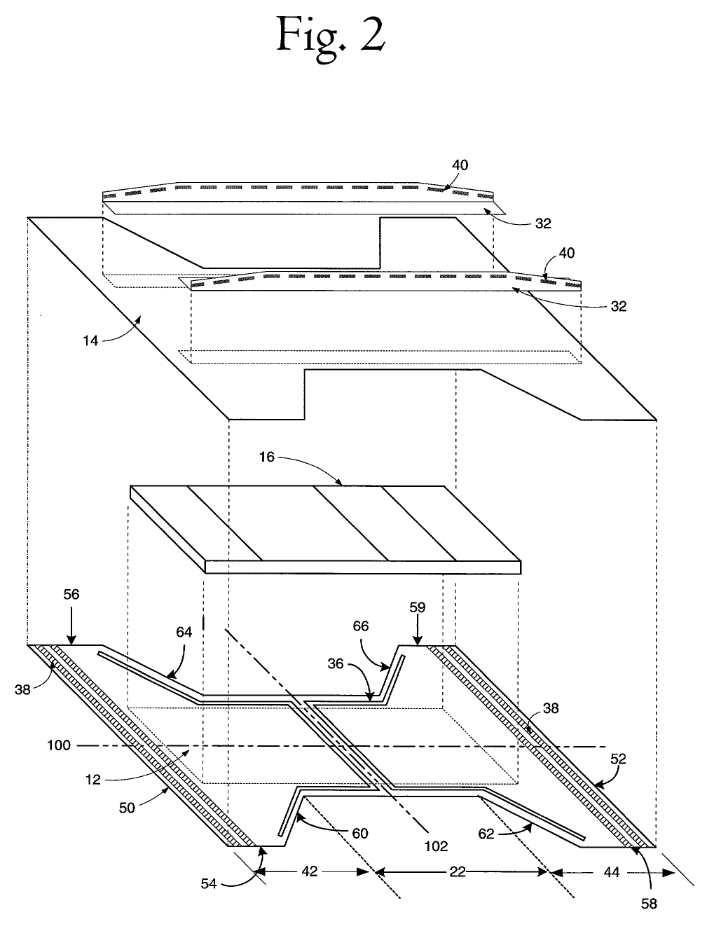

FIG. 2 is an exploded view of an embodiment of the present invention with elastic members shown in the elongated position for clarity, and the garment laid flat. The garment 10 has a longitudinal axis 100 corresponding approximately to the rear-to-front axis of the wearer, and a lateral axis 102, orthogonal to the longitudinal axis 100, and corresponding approximately to the side-to-side axis of the wearer. In one embodiment of the invention the lateral axis 102 of the garment 10 is approximately parallel with the machine direction of the garment 10.

In the embodiment of the invention depicted in FIG. 2, the main body of the garment comprises a back sheet 12 and top sheet 14 having substantially identical dimensions. Those skilled in the art will recognize, however, that back sheet 12 and top sheet 14 need not have substantially identical dimensions; rather, either material may be smaller or larger than the other. The perimeter of the main body of the garment is defined by a laterally extending front waist edge 50, a laterally extending rear waist edge 52, a longitudinally extending left front side edge 54 and left rear side edge 58, a longitudinally extending right front side edge 56 and right rear side edge 59, left front arcuate edge 60 and left rear arcuate edge 62 joining the longitudinally extending left front side edge 54 to the longitudinally extending left rear side edge 58, and right front arcuate edge 64 and right rear arcuate edge 66 joining the longitudinally extending right front side edge 56 to the longitudinally extending right rear side 59. The laterally extending front waist edge 50, left and right front side edges 54, 56, and at least part of left and right front arcuate edges 60, 64 form a front waist region 42. At least a portion of the left and right front arcuate edges 60, 64, and at least a portion of the left and right rear arcuate edges 62, 66, form a crotch region 22. The laterally extending rear waist edge 52, left and right rear side edges 58, 59, and at least part of left and right rear arcuate edges 62, 66 form a rear waist region 44.

Throughout this description, the terms “front,” “rear,” “left,” and “right” merely denote location relationships for purposes of explanation and clarity, and they generally relate to the location depicted in the drawings. Those skilled in the art appreciate that the front and rear of the absorbent garment may be reversed, as well as the left and right sides of the absorbent garment 10 depending upon the vantage point of the viewer.

The top sheet 14 and back sheet 12 may be constructed from a wide variety of materials known in the art. The invention is not intended to be limited to any specific materials for these components. The top sheet 14 and back sheet 12 can be shaped and sized according to the requirements of each of the various types of absorbent garment, or to accommodate various user sizes. In an embodiment of the invention in which the garment 10 is a diaper or an adult incontinence brief, the combination of top sheet 14 and back sheet 12, may have an hourglass shape, as seen in FIG. 1, or may have a rectangular, trapezoidal, “T” shape, or other shape.

Due to the wide variety of backing and liner sheet construction and materials currently available, the invention is not intended to be limited to any specific materials or constructions of these components. The back sheet 12 preferably is made from any suitable pliable liquid-impervious material known in the art. The selection and manufacture of such materials is well known in the art, and is disclosed, for example, in U.S. Pat. No. 6,123,694 issued to Peniak et al., and U.S. Pat. No. 6,176,952 issued to Maugans et al., the disclosure of each of which is incorporated herein by reference in its entirety, and in a manner consistent with the present invention. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The back sheet 12 may be pigmented with, for example, titanium dioxide, to provide the garment 10 with a pleasing color or to render the back sheet 12 opaque enough that exudates being contained by the garment 10 are not visible from outside the garment. In addition, the back sheet 12 may be formed in such a manner that it is opaque, for example, by using various inert components in the polymeric film and then biaxially stretching the film. Other back sheet materials will be readily apparent to those skilled in the art. The back sheet 12 preferably has sufficient liquid imperviousness to prevent any leakage of fluids. The required level of liquid imperviousness may vary between different locations on the garment 10.

The back sheet 12 may further comprise separate regions having different properties. In a preferred embodiment, portions of the back sheet 12 are air-permeable to improve the breathability, and therefore comfort, of the garment 10. The different regions may be formed by making the back sheet 12 a composite of different sheet materials, chemical treatment, heat treatment, or other processes or methods known in the art. Some regions of the back sheet 12 may be fluid pervious. In one embodiment of the invention, the back sheet 12 is fluid impervious in the crotch region 22, but is fluid pervious in portions of the first and second waist regions 42,44. The back sheet 12 may also be made from a laminate of overlaid sheets of material.

The moisture-pervious top sheet 14 can be comprised of any suitable relatively liquid-pervious material known in the art that permits passage of liquid there through. Non-woven liner sheet materials are exemplary because such materials readily allow the passage of liquids to the underlying absorbent core 16. Examples of suitable liner sheet materials include non-woven spunbond or carded webs of polypropylene, polyethylene, nylon, polyester and blends of these materials.

The back sheet 12 may be covered with a fibrous, nonwoven fabric such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded nonwoven web of synthetic fibers such as polypropylene, polyethylene or polyester fibers; a nonwoven web of cellulosic fibers, textile fibers such as rayon fibers, cotton and the like, or a blend of cellulosic and textile fibers; a spun-bonded nonwoven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the back sheet 12 may comprise three panels wherein a central poly back sheet panel is positioned closest to absorbent core 16 while outboard non-woven breathable side back sheet panels are attached to the side edges of the central poly back sheet panel. Alternatively, the back sheet 12 may be formed from microporous poly coverstock for added breathability.

Although not illustrated in the drawings, the top sheet 12 may be formed of three separate portions or panels. Such an embodiment is disclosed, for example, in U.S. Pat. No. 5,275,590 issued to Huffman et al., which is incorporated herein by reference in its entirety, and in a manner consistent with the present invention. Those skilled in the art will recognize, however, that top sheet 12 need not be made of three separate panels, and that it may be comprised of one unitary item, or of a top sheet material 14, with separate standing leg gathers material 32 attached thereto, as shown in FIG. 2. A first top sheet panel may comprise a central top sheet panel formed from preferably a liquid-pervious material that is either hydrophobic or hydrophilic. The central top sheet panel may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel is a cover stock of single ply non-woven material which may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine direction strength suitable for use as a baby diaper cover stock material. The central top sheet panel preferably extends from substantially the second waist region 44 to the first waist region 42, or a portion thereof.

The second and third top sheet panels (e.g., outer top sheet panels), in this alternative embodiment may be positioned laterally outside of the central top sheet panel. The outer top sheet panels preferably are substantially liquid-impervious and hydrophobic, preferably at least in the crotch area. The outer edges of the outer top sheet panels may substantially follow the corresponding outer perimeter of the back sheet 12. The material for the outer top sheet portions or panels is preferably polypropylene and can be woven, non-woven, spunbonded, carded or the like, depending on the application.

The respective top sheet panels may be attached to one another by, e.g., an adhesive. At the point of connection with the outer edges of the central top sheet panel and the inner edges of the outer top sheet panels extend upwardly to form waste containment flaps 32 (waist containment flaps 32, or standing leg gathers 32, are shown as separate elements in FIG. 2, but they need not be). The waste containment flaps 32 preferably are formed of the same material as the outer top sheet panels, as in the embodiment shown. They most preferably are an extension of the outer top sheet panels.

The waste containment flaps 32 may be treated with a suitable surfactant to modify their hydrophobicity/hydrophilicity as desired, and they may be treated with skin wellness ingredients to reduce skin irritation. Alternatively, the waste containment flaps 32 may be formed as separate elements and then attached to the body side liner. In this alternative embodiment, the central top sheet panel may extend past the connection point with the waste containment flaps 34, and even extend to the periphery of the back sheet 12.

The waste containment flaps 32 preferably include a portion that folds over onto itself to form a small enclosure. At least one, and depending on the size of the enclosure sometimes more than one, elastic element 40 may be secured in the enclosure in a stretched condition. As has been known at least as long the disclosure of Tetsujiro, Japanese Patent document 40-11543, when the flap elastic element 40 attempts to assume the relaxed, unstretched condition, the waste containment flaps 34 rise above the surface of the center of the top sheet 14, as shown in FIG. 2.

The top sheet 14 may be made of any suitable relatively liquid-pervious material currently known in the art or later discovered that permits passage of a liquid there through. Examples of suitable top sheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent core 16. The top sheet 14 preferably comprises a single-ply nonwoven material that may be made of carded fibers, either adhesively or thermally bonded, spunbonded fibers, or water entangled fibers, which generally weigh from 0.3-0.7 oz./sq. yd. and have appropriate and effective machine direction (longitudinal) and cross-machine (lateral) direction strength suitable for use as a top sheet material for the given application. The present invention is not intended to be limited to any particular material for the top sheet 14, and other top sheet materials will be readily apparent to those skilled in the art.

The top sheet 14 may further comprise several regions having different properties. In one embodiment of the present invention, the laterally distal portions of the top sheet 14, especially those used to make second and third top sheet panels, preferably are substantially fluid impervious and hydrophobic, while the remainder of the top sheet 14 (e.g., central top sheet panel) is hydrophilic and fluid pervious. Different top sheet properties, such as fluid perviousness and hydrophobicity, may be imparted upon the top sheet 14 by treating the top sheet 14 with adhesives, surfactants, or other chemicals, using a composite of different materials, or by other means. The top sheet 14 may also be made from a laminate of overlaid sheets of material. The top sheet 14 also may be treated in specific areas like the crotch region, with skin wellness ingredients such as aloe, vitamin E, and the like.

As noted elsewhere herein, the top sheet 14 and back sheet 12 may be substantially coterminous, or they may have different shapes and sizes. The particular design of the top sheet 14 and back sheet 12 may be dictated by manufacturing considerations, cost considerations, and performance considerations. Preferably, the top sheet 14 is large enough to completely cover the absorbent core 16, and the back sheet 12 is large enough to prevent leakage from the garment 10. The design of top sheet 14 and back sheet 12 is known in the art, and a skilled artisan will be able to produce an appropriate top sheet 14 and an appropriate back sheet 12 without undue experimentation.

The top sheet 14 and the back sheet 12 may be associated with one another using a variety of methods known in the art. For example, they may be thermally, ultrasonically, or chemically bonded to one another. They also may be joined using lines of hot melt adhesive or mechanical fasteners, 15 such as thread, clips, or staples. In one embodiment, a hydrophilic adhesive, such as Cycloflex as sold by National Starch, a corporation headquartered in Bridgewater, N.J., is used to join the top sheet 14 to the back sheet 12. The particular joining method may be dictated by the types of materials selected for the top sheet 14 and back sheet 12.

In one embodiment of the present invention, the top sheet 14 is operatively associated with the back sheet 12 around the perimeter of the top sheet 14. In this embodiment, the top sheet 14 and back sheet 12 may be operatively associated with one another by using hot melt adhesives, ultrasonic bonding, or any other suitable method known in the art. Also in this embodiment, the top sheet 14 and back sheet 12 may be bonded to one another in substantially all areas not having intermediately placed parts, such that some or all of the intermediately placed, or “sandwiched,” parts are physically captured between the top sheet 14 and back sheet 12, but not bonded to the back sheet 12 or top sheet 14.

An absorbent core 16 preferably is disposed between the inner surfaces of the back sheet 12 and the top sheet 14. The absorbent core 16 may be comprised of one or more layers of material, such as an absorbent layer for storing fluids and an acquisition layer for distributing fluids. Such multiple layer absorbent cores are known in the art and disclosed in U.S. Pat. No. 5,439,458 issued to Noel et al., which is incorporated herein by reference in its entirety, and in a manner consistent with the present invention.

The absorbent core 16 may be made from any absorbent material or materials known in the art. In one embodiment of the invention, the absorbent core 16 comprises wood fibers or other fibers such as chemical wood pulp, or any other suitable liquid absorbing material, such as commercially available fluff pulp or fluffed bleached kraft softwood pulp. In another embodiment of the invention, the absorbent core 16 comprises a combination of a porous fibrous web and super absorbent particles. Such absorbent cores are known in the art and are disclosed, for example, in U.S. Pat. No. 5,281,207 issued to Chmielewski et al., which is incorporated herein by reference in its entirety, and in a manner consistent with the present invention. In such an embodiment, the absorbent core 16 may be surrounded by a liquid pervious tissue over-wrap, or other material.

The absorbent core 16 also may be a laminate material, as described in U.S. Pat. No. 6,068,620, the disclosure of which is incorporated by reference herein in its entirety, and in a manner consistent with this disclosure. Such laminate materials typically include outer tissue layers surrounding a central fibrous layer that contains a high percentage by weight of superabsorbent polymer (SAP). Any type of SAP can be used in this embodiment, or in any absorbent core 16 that is useful in this invention. The SAP generally is a water-insoluble but water-swellable polymeric substance capable of absorbing water in an amount which is at least ten times the weight of the substance in its dry form. In one type of superabsorbent material, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

The absorbent core 16 generally is elongated along the longitudinal axis 100 of the garment, and may extend along either or both of the lateral and longitudinal axes 102, 100 to the outer perimeter of the garment. In the embodiment depicted in FIG. 2, the absorbent core 16 is substantially rectangular in shape, however, it may also have rounded ends or other shapes, such as an “I” shape or a “T” shape. The absorbent core 16 may also have channels, grooves or pockets, and may have a varying thickness.

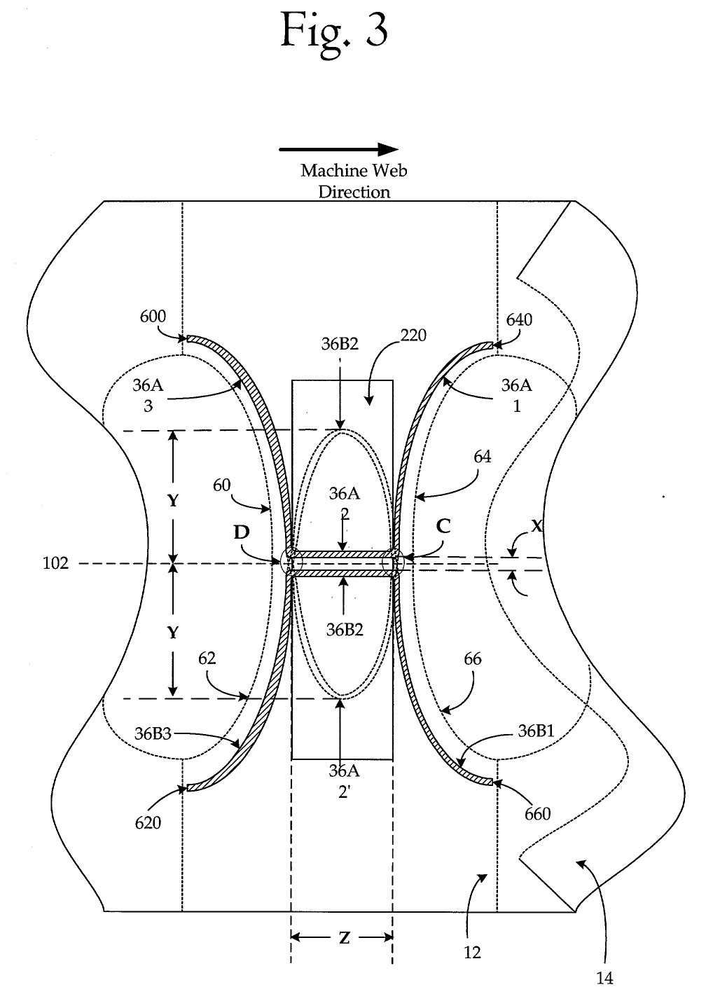

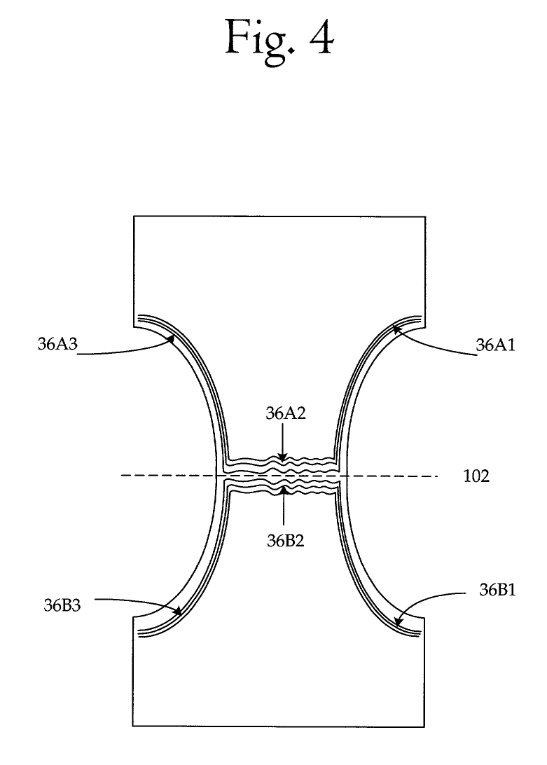

The garment 10 of the present invention may include various mechanisms for improving the fit of the garment 10 to the wearer, and for improving leakage resistance. In one embodiment of the invention, leg gathers 36 are used to contract the leg holes 28 of the garment 10 around the wearer's legs to prevent body waste from escaping. In a preferred embodiment, leg gathers 36 are incorporated into the garment 10 by extending one or more pieces of elastic and adhering them to an element of the garment, preferably to certain portions of the top sheet 14, back sheet 12, or other components while the elastic is in an elongated state. When the elastic contracts, the parts of the garment to which the elastic elements are attached constrict around the user's body. A more detailed description of the leg gathers 36, and how they are incorporated into garment 10 is included below with reference to FIGS. 3 and 4. The elastic elements may be made from natural rubber, lycra, polyurethane, heat shrinkable polymer ribbons, or any other suitable elastic material or composite. Leg gathers are known in the art, and U.S. Pat. No. 5,660,664 issued to Hermann, which is incorporated herein by reference in its entirety, and in a manner consistent with the present invention, discloses an exemplary method of manufacturing leg gathers.

In another embodiment of the invention the garment 10 is equipped with standing leg gathers 32. Standing leg gathers 32 generally are comprised of flaps or sleeves of liquid impervious material that have one or more elastic elements 40 in them. The flaps are drawn against the user's body by the contracting force of the elastic 40, and help seal the garment 10 against leakage. The elastic elements 40 may comprise any suitable elastic material, and the flap portions of the leg gathers 32 may be made as an integral part of the top sheet 14 or back sheet 12, or from additional fabric material that may or may not be liquid pervious, as shown in FIG. 2. Standing gathers are disclosed, for example, in U.S. Pat. No. 5,292,316 issued to Suzuki, which is incorporated herein by reference in its entirety, and in a manner consistent with the present invention.

The absorbent garment 10 of the invention also preferably is provided with waist elastic material 38 optionally in the first and second waist regions, 42, 44, respectively, to enable and assist in stretching around the wearer. The waist elastics 38 may be similar structures or different to impart similar or different elastic characteristics to the first and second waist regions 42,44 of the garment. In general, the waist elastics may preferably comprise foam strips positioned at the first and second waist regions 42, 44, respectively. Such foam strips preferably are about ½ to about 1 ½ inches wide and about 3-6 inches long. The foam strips preferably are positioned between the top sheet portions 14 (or panels) and the back sheet 12. Alternatively, a plurality of elastic strands may be employed as waist elastics 38 rather than foam strips. The foam strips preferably are comprised of polyurethane, but can be any other suitable material that decreases waist band roll over, reduces leakage over the waist ends of the absorbent garment, and generally improve comfort and fit. The first and optional second waist foam strips 38 preferably are stretched 5-300%, preferably 50 to 150%, and more preferably, about 100% more than their unstretched dimension before being adhesively secured between the back sheet 12 and top sheet 14.

The elastic elements 36, 38, and 40 may be selected to provide a greater or lesser contracting force for different parts of the garment 10. For example, elastics having a high contracting force may be selected for the regions of the leg gathers nearest the bottom portion of leg hole 28 (e.g., center portions of arcuate side edges 60 and 62, or 64 and 66). In addition, elastics having a lesser contracting force may be selected for those parts of the leg gathers around the upper portions of leg holes 28 (e.g., near the front and rear waist region 42, 44). Such differences in contracting force may be obtained by providing elastic elements 36, 40 having differing cross sectional shapes, by providing elastic elements 36, 40 made from different materials, by providing a greater or lesser number of elastic elements 36, 40 or by any other method known in the art. In the embodiment of the invention depicted in FIG. 2, there also are a plurality of waist elastics 38, providing a greater contracting force near the user's waist. Skilled artisans recognize that the waist elastic elements 38 are optional, and that one or more elastic elements 38 may be disposed in first and second waist regions 42, 44.

The absorbent garment 10 of the invention also preferably includes a fastening mechanism by which the front waist region 42 is associated with rear waist region 44 to form a waist hole 30. The fastening mechanism may comprise a permanent seal whereby left front side edge 54 is attached to left rear side edge 58, and right front side edge 56 is attached to right rear side edge 59 to form side seals 26 (FIG. 1). The absorbent garment 10 then can be pulled on and off like an undergarment or pair of pants. Any mechanism can be used to form the permanent seal 26. The use of the expression “permanent seal” in this context is not meant to encompass seals that cannot be broken, but rather permanent is meant to encompass sealing mechanisms that are not intended to be broken during normal use and application. The respective side edges can be associated with one another to form seal 26 using techniques known in the art, including, for example, using adhesives such as hot melt adhesives and construction adhesives, chemical or solvent bonding, ultrasonic welding, stitching, heat bonding, or any other method of affixation known or hereafter discovered.

The absorbent garment 10 of the invention may also include a releasable fastening mechanism by which the front waist region 42 is associated with rear waist region 44 to form a waist hole 30. The releasable fastening mechanism may comprise a mechanism whereby left front side edge 54 is releasably attached to left rear side edge 58, and right front side edge 56 is releasably attached to right rear side edge 59 to form side seals 26 (FIG. 1).

Releasable fastening mechanisms are well known in the art, and may include tabs laterally extending from the laterally opposing rear side edges 58, 59, adhesive strips, belts, and the like. The particular mechanism by which the respective parts of garment 10 may be adhered to one another include hook and loop type fasteners, pressure sensitive adhesives, snaps, clips, pins, and the like. Those skilled in the art are capable of manufacturing garment 10 to have either permanent or releasable fastening mechanisms, using the guidelines provided herein.

Referring now to FIG. 3, it is preferred in the invention that the leg elastics 36 be applied to the garment 10 in such a manner that no gathering or shirring of the top sheet or back sheet occurs in lateral center portion of the crotch region 22 (denoted by the shaded rectangle 220 in FIG. 3). It also is preferred that the elastic elements 36 in this area of the garment not be under any tensile load. That is, elastic elements 36 in the lateral center portion of the crotch region 22 should hang freely, and should not be in contact with elastic elements on the opposite side of lateral centerline 102, as shown more clearly in FIG. 4.

The elastic elements 36 preferably are applied to a moving web, or webs, during manufacture of the absorbent article. Preferably, the top sheet 14, back sheet 12, and absorbent core 16 are applied in a continuous manner to a forming station whereby all of the respective elements are brought into contact with one another and pressed together to form the ultimate absorbent garment 10. The top sheet 14 is cut away in FIG. 3 to reveal the respective elements of the garment. The absorbent core 16 also has been removed for purposes of clarity. The elastic elements 36, and 38 preferably are supplied to the forming station along with the top sheet 14, back sheet 12, and absorbent core 16. Supplying elastic elements to a forming station during manufacture of an absorbent article is well known in the art, and is described in, for example, U.S. Pat. Nos. 5,147,487, 5,188,627, 5,745,922, 6,098,203, and RE 37,154E, the disclosures and drawings of which are incorporated by reference herein in their entirety, and in a manner consistent with this disclosure. Any of the methods for supplying elastic elements to a forming station disclosed therein are suitable in the present invention.

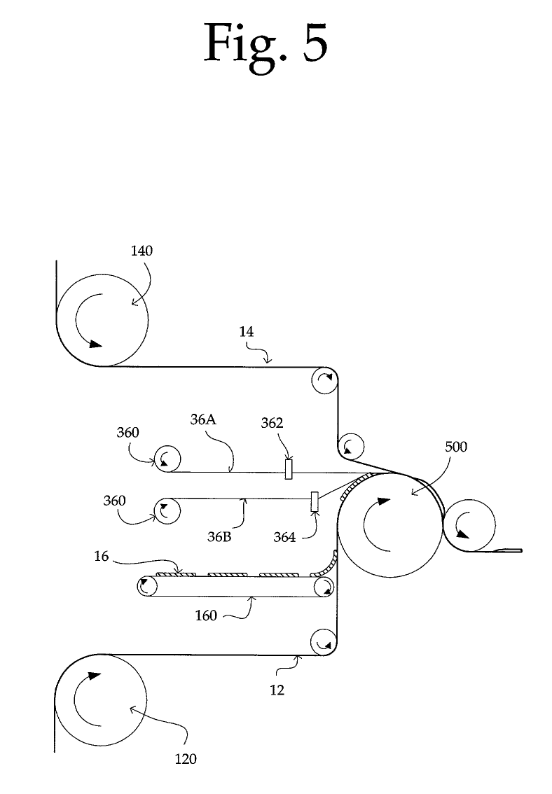

Referring again to FIG. 3, the various components are supplied in the machine web direction, which is shown by the right arrow. When elastic elements 36 are supplied to the moving webs, the shape of the garment 10 has not yet been cut to form arcuate edges 60, 62, 64, and 66. Hence, these side edges are denoted by the dotted lines in FIG. 3. Applying elastic elements 36 to the moving web can be accomplished by moving the elastic element supply mechanisms (362,364 in FIG. 5) in a direction orthogonal to the machine web direction. Hence, the elastic element supply mechanisms would move back-and-forth along the longitudinal axis 100 of garment 10.

As the web(s) is/are moving in the machine web direction, a first elastic element supply mechanism 362 will initially be disposed at or near the upper portion 640 of right front arcuate side edge 64. The first elastic element supply mechanism 362 then would move longitudinally down the garment toward the lateral centerline 102 at such a rate relative to the rate the web(s) is/are moving in the machine web direction to deposit first elastic element segment 36A1 substantially adjacent to right front arcuate edge 64. Adhesive or other mechanism capable of adhering elastics to garment 10, preferably is applied either to one of the moving webs (e.g., either the top sheet 14, back sheet 12, or other associated element), or an adhesive is applied to the elastic element to allow first elastic element segment 36A1 to adhere to garment 10 at the forming station. Applying adhesives to the various elements of the absorbent garment 10 is well known in the art, and a skilled artisan is capable of designing a suitable adhesive applicator for use in the invention, using the guidelines provided herein.

The first elastic element supply mechanism 362 then continues past lateral centerline 102 and into the rear region of garment 10, as denoted by the dotted arc 36A2′. When the elastic element supply mechanism reaches a distance Y from the lateral centerline 102, it reverses its longitudinal orientation and begins moving longitudinally back toward the lateral centerline 102, thereby completing the dotted arc 36A2′. The total lateral distance traveled by elastic element supply mechanism in forming second elastic element segment 36A2 is denoted by the letter Z in FIG. 3. Because elastic element 36 is supplied to the garment under tension, because elastic element 36 is overstretched to a distance Y, and because there is no adhesive in the crotch region 220 denoted by the shaded area, the second elastic element segment 36A2 will be formed in such a manner that it snaps back to its originating side of lateral centerline 102 (the front side in FIG. 3), and hangs loosely as shown in FIG. 4. No gathering or shirring of the absorbent garment will occur in the crotch region 220, and substantially no cross core compression will occur.

The first elastic element supply mechanism 362 then will travel in a longitudinal direction past centerline 102 and back into the front region of garment 10 toward the front waist edge. This movement will deposit the third elastic element segment 36A3 substantially along left front arcuate edge 60, terminating at or near the upper portion 600 of left front arcuate edge 60. Adhesive or other joining mechanism again preferably is applied in this region of the garment in a manner similar to that used to form first elastic element segment 36A1, thereby adhering third elastic element segment 36A3 to garment 10.

The first elastic element supply mechanism 362 travels in the arcuate path denoted by dotted line 36A2′ by traveling a longitudinal distance Y from centerline 102, and a lateral distance Z, which corresponds substantially to the lateral width of crotch region 220. The shape of the movement of the first elastic element supply mechanism 362, with respect to the moving web(s) is sinusoidal, thereby making Y the amplitude, and Z the frequency of elastic element supply mechanism 362's motion. It is a feature of a preferred embodiment of the invention that the amplitude (Y) divided by the frequency (Z) be such that no tension is created in second elastic element segment 36A2. Accordingly, second elastic element segment 36A2 will not pull to any appreciably extent first and third elastic element segments 36A1, 36A3 at position C and D, toward the longitudinal centerline 100 of garment 10. As a consequence, no shirring or gathering of material will occur in this region of the garment, thereby making the garment less irritating to the wearer, and more aesthetically pleasing. Because second elastic element segment 36A2 is not under tension, tearing or displacement of first and third elastic element segments 36A1, 36A3 is reduced or eliminated at positions C and D. In addition, providing second elastic element segment 36A2 in this manner significantly reduces, and may eliminate, cross core compression, thereby increasing the capacity of the absorbent core 16.

It is preferred in the present invention that Y vary anywhere from about 30 mm to about 280 mm, preferably from about 95 mm to about 225 mm, and most preferably from about 125 mm to about 175 mm. It also is preferred that Z vary anywhere from about 50 mm to about 250 mm, preferably from about 75 mm to about 175 mm, and most preferably from about 100 mm to about 150 mm. It also is preferred that Y/Z be within the range of from about 0.1 to about 10, preferably from about 0.5 to about 5, and most preferably about 1 to about 3.

Another longitudinally opposing elastic element, 36B can be applied to the other longitudinal side (e.g., rear) of garment 10 in a similar manner as that described above. Specifically, as the web(s) is/are moving in the machine web direction, second elastic element supply mechanism 364 (FIG. 5) initially will be disposed at or near the lower portion 660 of right rear arcuate side edge 66 (e.g., the machine direction leading edge). The second elastic element supply mechanism 364 then would move longitudinally up the garment toward the lateral centerline 102 at such a rate relative to the rate the web(s) is/are moving in the machine web direction to deposit first elastic element segment 36B1 substantially adjacent to right rear arcuate edge 66. Adhesive or other mechanism capable of adhering elastic elements to garment 10, preferably is applied either to one of the moving webs (e.g., either the top sheet 14, back sheet 12, or other associated element), or the adhesive is applied to the elastic element to allow first elastic element segment 36B1 to adhere to garment 10 at the forming station. Applying adhesives to the various components of the absorbent garment 10 is well known in the art, and a skilled artisan is capable of designing a suitable adhesive applicator for use in the invention, using the guidelines provided herein.

The second elastic element supply mechanism 364 then continues past lateral centerline 102 and into the front region of garment 10, as denoted by the dotted arc 36B2′. When the elastic element supply mechanism reaches a distance Y from the lateral centerline 102, it reverses its longitudinal orientation and begins moving longitudinally back toward the lateral centerline 102, thereby completing the dotted arc 36B2′. The total lateral distance traveled by the second elastic element supply mechanism 364 in forming second elastic element segment 36B2 is denoted by the letter Z in FIG. 3. Because elastic element 36B2 is supplied to the garment under tension, because elastic element 36B2 is overstretched to a distance Y, and because there is no adhesive in the crotch region 220 denoted by the shaded area, the second elastic element segment 36B2 will be formed in such a manner that it snaps back to its originating side of lateral centerline 102 (the rear side in FIG. 3), and hangs loosely as shown in FIG. 4. No gathering or shirring of the absorbent garment will occur in the crotch region 220, and substantially no cross core compression will occur.

The second elastic element supply mechanism 364 then will travel in a longitudinal direction past centerline 102 and back into the rear region of garment 10 toward the rear waist edge. This movement will deposit the third elastic element segment 36B3 substantially along left rear arcuate edge 62, terminating at or near the lower portion 620 of left rear arcuate edge 62. Adhesive or other joining mechanism again preferably is applied in this region of the garment in a manner similar to that used to form first elastic element segment 36B1, thereby adhering third elastic element segment 36B3 to garment 10.

Those skilled in the art will recognize that first and second elastic element supply mechanisms 362, 364 will need to crisscross their respective paths during application of elastic element(s) 36. That is, when elastic element 36A is being formed, the first elastic element supply mechanism 362 will have to cross over the second elastic element supply mechanism 364 used to form elastic element 36B, at points C and D in FIG. 3. It is therefore preferred in the invention to stagger slightly the application of elastic elements 36A and 36B. Accordingly, the first elastic element supply mechanism 362 used to supply elastic element 36A may be disposed upstream in the machine web direction from the second elastic element supply mechanism 364 used to supply elastic element 36B, as shown more clearly in FIG. 5. Other techniques known to skilled artisans also could be used to supply elastic elements 36A and 36B in the manner illustrated in FIGS. 3 and 5.

Supplying elastic elements 36A and 36B in the manner shown in FIG. 3 also enables very close placement of these elastic elements in crotch region 220, as denoted by the distance “X” in FIG. 3. It is preferred in the present invention that the longitudinal distance (i.e., X) between the elastic elements 36 disposed in the front region of garment 10 (e.g., elastic element 36A) and the elastic elements 36 disposed in the rear region of garment 10 (e.g., elastic element 36B) be anywhere from about 0.1 to about 50 mm, preferably from about 0.5 to about 15 mm, and most preferably from about 0.75 to about 5 mm. Those skilled in the art are capable of controlling the various variables in making absorbent garment 10 to enable such close placement of elastic elements 36, using the guidelines provided herein.

More than one pair of leg elastics 36 may be supplied to garment 10, as will be appreciated by those skilled in the art. It is preferred that up to about 3 or 4 pair of leg elastics 36 be applied, as shown in FIG. 4. FIG. 4 also shows leg elastics 36 being under substantially no tensile load in the crotch region 220, as indicated by the wavy lines 36A2 and 36B2.

The invention also relates to a method of forming an absorbent garment that includes providing a back sheet material 12 and a top sheet material 14 to a forming station 500, as shown in FIG. 5. The method also includes providing an absorbent core 16 and at least one pair of leg elastics 36A, 36B to the forming station 500 and disposing the absorbent core and the at least one pair of leg elastics between the back sheet material 12 and the top sheet material 14. The absorbent garment so formed then is cut to form a final absorbent garment having the following characteristics:

a longitudinal dimension corresponding to the front and back of a wearer, and a lateral dimension corresponding to the left and right side of a wearer, whereby the lateral dimension corresponds substantially to the machine direction of the forming station, and the longitudinal dimension is orthogonal to the page in FIG. 5;

laterally extending front and rear waist edges 50, 52 (see FIG. 2), and a pair of left and right side edges, where at least the laterally extending front waist edge 50, the left front side edge 54, and right front side edge 56 form a front waist region 42. In a similar fashion, at least the laterally extending rear waist edge 52, the left rear side edge 58, and the right rear side edge 59 form a rear waist region 44;

left front arcuate edge 60 and left rear arcuate edge 62 joining the left front side edge 54 to the left rear side edge 58, and right front arcuate edge 64 and right rear arcuate edge 66 joining the right front side edge 56 to the right rear side edge 59, whereby at least part of the arcuate edges 60, 62, 64, 66 form a crotch region 22 that is disposed between the front and rear waist regions, 42, 44. When the front and rear waist regions 42, 44 are brought together, the arcuate edges define leg openings 28 (FIG. 1).

Providing the at least one pair of longitudinally opposing leg elastics 36A, 36B comprises supplying at least one elastic element 36A to the front half of the garment via first elastic element supply mechanism 362, and at least one elastic element 36B to the rear half of the garment via second elastic element supply mechanism 364, whereby the elastic elements are supplied in a staggered fashion such that the second elastic element supply mechanism 364 is disposed closer to forming station 500 than first elastic element supply mechanism 362. While elastic elements 36A, 36B are supplied to the front and rear halves of the garment in a staggered fashion, they are supplied in a similar manner, albeit one after the other on opposing sides of the lateral centerline 102 of the garment.

Supplying the at least one elastic element 36A, 36B to the front or rear half of the garment comprises supplying from a continuous elastic element supply roller 360 the elastic elements in a sinusoidal configuration so that the elastic element includes three different segments. The first elastic element segments 36A1, 36B1 (see, FIG. 3) are disposed adjacent the front or rear arcuate edges 64, 66 of the garment leading in the machine direction, the second elastic element segments 36A2, 36B2 extend laterally across the crotch region 220 of the garment, and the third elastic element segments 36A3, 36B3 are disposed adjacent the longitudinally opposing front or rear arcuate edges 60, 62 of the garment trailing in the machine direction. The leg elastics 36A disposed in the longitudinal front portion of the garment do not contact the leg elastics 36B disposed in the longitudinal rear portion of the garment, and they preferably are separated from one another by a distance of from about 0.1 mm to about 50 mm. The first and third segments of the longitudinally opposing leg elastics are adhered to at least one element of the garment (e.g., the top sheet 14 or the back sheet 12), under tension, and the second segment of the longitudinally opposing leg elastic elements 36A2, 36B2 is applied in such a fashion it is are not adhered to another element under tension, and such that they are under no tensile load after application. Thus, second elastic element segments 36A2, 36B2 may be adhered to an element of the garment after they have been applied and snapped back to their respective relaxed positions (FIG. 4). It is preferred, however, that second elastic element segments 36A2, 36B2 are not adhered to any element of the garment.

FIG. 5 depicts an embodiment of a manufacturing process for producing a garment 10 having the desired features of the present invention. The various parts of the garment are described in more detail elsewhere herein.

In the preferred embodiment depicted in FIG. 5, a continuous supply of back sheet material 12 is provided along the machine direction to forming station 500 via back sheet supply roller 120. Absorbent cores 16 preferably are deposited on the back sheet material 12 (or another layer or layer(s) interposed there between) on the surface that will eventually become the inner surface of the back sheet 12 of the garment 10. The absorbent cores 16 may be bonded or otherwise operatively associated with the back sheet material 12 at this point or thereafter. Skilled artisans also will recognize that absorbent cores need not be bonded or operatively associated with back sheet material 12, or top sheet material 14, and that other layers may be present between back sheet 12 and absorbent core 16.

A continuous supply of top sheet material 14 is provided substantially in the machine direction to forming station 500 via top sheet supply roller 140. The supply of top sheet material 14 preferably overlays the back sheet material 12 and encases the absorbent cores 16. The top sheet material 14 may be adhesively bonded to, or otherwise operatively associated with, the back sheet material 12 or other parts of the assembly at this location or hereafter. The method also includes providing to forming station 500, at least a pair, e.g., at least two, continuous supplies of standing leg gather elastic elements 36A, 36B via elastic element supply rollers 360 in substantially the machine direction.

The elastic elements 36A are provided to forming station in the manner discussed above (e.g., in a sinusoidal fashion) by moving the first elastic element supply mechanism 362 back and forth in a direction orthogonal to the machine direction (i.e., into and out of the page). It is preferred that the elastic elements 36A, 36B are stretched or extended while providing them to forming station 500, and more preferably, they are extended to between approximately 100% and approximately 350% of their relaxed length. The elastic elements 36A are adhered to an element of the garment, e.g., to the top sheet material 14 or back sheet material 12 near the arcuate edges 60, 64, but preferably are not adhered to another element of the garment, e.g., not adhered to either the top sheet material 14 or back sheet material 12 in the central crotch region 220 (FIG. 3). Elastic elements preferably are not, although they may be, attached to absorbent core 16 in the central crotch region 220.

The elastic elements 36B are provided to forming station 500 in a similar manner as elastic elements 36A, only on the longitudinally opposing side of garment 10. Elastic elements 36 B are provided to forming station 500 preferably in a sinusoidal fashion by moving the second elastic element supply mechanism 364 back and forth in a direction orthogonal to the machine direction (i.e., into and out of the page). The elastic elements 36B may be adhered to an element of the garment, e.g., to either the top sheet material 14 or back sheet material 12 near the arcuate edges 62, 66, but preferably are not adhered to another element of the garment, e.g., not adhered to either the top sheet material 14 or back sheet material 12 in the central crotch region 220 (FIG. 3). Elastic elements preferably are not, although they may be, attached to absorbent core 16 in the central crotch region 220. Because the first and second elastic element supply mechanisms 362,364, will crisscross one-another at positions C and D in FIG. 3, it is preferred that one of the supply mechanisms (e.g., 364) be disposed closer to the forming station 500 than the other.

Any elastic element supply mechanism 362, 364 can be used to supply elastic elements 36A, 36B to forming station 500. Suitable elastic element supply mechanisms are disclosed in, for example, U.S. Pat. Nos. 5,147,487, 5,188,627, 5,745,922, 6,098,203, and RE 37,154E, the disclosures and drawings of which are incorporated by reference herein in their entirety, and in a manner consistent with this disclosure. Any of the methods for supplying elastic elements to a forming station disclosed therein are suitable in the present invention.

The respective component parts of the absorbent garment 10 are brought together and associated with one another at forming station 500. Here, any adhesive or other joining mechanism applied to back sheet material 12 or top sheet material 14 will serve to adhere the respective materials to one another. The same holds true for adhesive present at or near the absorbent core 16, and/or elastic elements 36A, 36B. In addition, other components may be included in the garment at this stage or later, and other processing steps may take place to form garment 10. For example, an additional ultrasonic bonding apparatus may be employed, additional adhesive applicators may be disposed to apply adhesive to top sheet 14, back sheet 12, leg elastic elements 36A, 3613, absorbent core 16, and the like.

After bringing the components together at forming station 500, the absorbent garment is further processed by cutting and folding, and/or carrying out other processing procedures (e.g., providing standing leg gathers 34, etc.). For example, the top sheet material 14 and back sheet material 12 preferably are cut to form leg-hole cutouts 28 on each side of the assembly. Any suitable cutting device may be used, such as fixed blades, cutting drums or reciprocating cutters. The top sheet material 14 and back sheet material 12, and any other components may also be operatively associated with one another during the same operation. For example, a single device may provide blades to cut the leg hole cutouts 28, and also provide anvils to form ultrasonic bonds or heated elements to activate adhesives previously deposited on one or more of the parts. Additional heating drums or pressure devices may be employed to provide suitable seals along the respective front, rear, left, and right side and arcuate edges of garment 10, and then the garment cut and severed from the moving web. Additional processing steps also may take place to fasten the laterally extending front and rear waist edges 50, 52 to one another. For example, the garment 10 may be folded in half, and then adhesives, ultrasonic welds, and the like used to join the left front side edge 54 with the left rear side edge 58, and to join the right front side edge 56 with the right rear side edge 59, thereby forming a disposable pants type garment 10, such as that shown in FIG. 1. Alternatively, tabs (not shown) may be attached to or integrally formed with, top sheet material 14 or back sheet material 12 at the left and right rear side edges 58, 59, whereby the tabs support fastening elements. Any fastening elements may be used, including, inter alia, hooks, loops, tapes, clips, snaps, and the like. At least one target device then can be disposed at or near the front waist region 42 of the garment whereby the at least one target devices forms a landing zone and otherwise cooperates or associates with the fastening elements. Suitable target devices include, hooks, loops, tape landing zones, clips, snaps, etc. Those skilled in the art are capable of associating the front waist region 42 with the rear waist region 44 to form waist opening 30, using the guidelines provided herein.

One skilled in the art will understand that the locations of the various parts of the invention during the assembly process, and the intervals at which parts are placed on the assembly, are selected such that the various parts are in their proper location in the final products. In addition, other parts, such as waist elastic elements 38 or standing leg gathers 32, may be incorporated into the assembly during the assembly process, and other processes, such as folding and packaging, may be incorporated into the assembly process. Also, it should be understood that any suitable method may be used to introduce the various parts to the assembly line, such as rollers, vacuum drums, or reciprocating stamps. Finally, it should also be understood that the order of the various forming procedures may be modified, combined, or rearranged to provide for various assembly sequences that will provide substantially the same finished product, and all such variations are within the scope and spirit of the present invention and are within the knowledge and skill of those skilled in the art in light of the present teachings. For example, application of top sheet material 14 may take place subsequent to, or just prior to forming station 500. Other modifications will be apparent to those skilled in the art.

Although the present invention has been described in terms of particular embodiments, it is not limited to these embodiments. Alternative embodiments and modifications that would still be encompassed by the invention may be made by those skilled in the art, particularly in light of the foregoing teachings. Therefore, the following claims are intended to cover any alternative embodiments, modifications or equivalents that may be within the spirit and scope of the invention.

We claim:

1. An absorbent garment comprising a main body including a top sheet and a back sheet, the garment having a longitudinal dimension corresponding to the front and back of a wearer, and a lateral dimension corresponding to the left and right side of a wearer; the garment comprising:
laterally extending front and rear waist edges, left and right front side edges, and left and right rear side edges, where at least the front waist edge and left and right front side edges form a front waist region, and at least the rear waist edge and the left and right rear side edges form a rear waist region;
a left front arcuate edge and a left rear arcuate edge joining the left front side edge to the left rear side edge;
a right front arcuate edge and a right rear arcuate edge joining the right front side edge to the right rear side edge, whereby at least part of the arcuate edges form a crotch region that is disposed between the front and rear waist regions, and when the front and rear waist regions are brought together, the left and right, front and rear arcuate edges define leg openings;
at least a pair of longitudinally opposing leg elastics that are disposed adjacent the left and right, front and rear, arcuate edges, and that extend laterally across the crotch region of the garment, whereby the leg elastics are comprised of at least three segments;
a) the first elastic element segment being disposed adjacent one front or rear arcuate edge of the garment;
b) the second elastic element segment extending laterally across the crotch region of the garment; and
c) the third elastic element segment is disposed adjacent the laterally opposing front or rear arcuate edge of the garment,
whereby the leg elastic elements disposed in the longitudinal front portion of the garment do not contact the leg elastics disposed in the longitudinal rear portion of the garment, the first and third elastic element segments are adhered to at least one other element of the garment under tension, and the second elastic element segment is not adhered to another element of the garment under tension, and whereby the leg elastic elements are applied to the garment in such a fashion that the second elastic element segment is under no tensile load after application.

2. The absorbent garment as claimed in claim 1, wherein the leg elastic elements disposed in the longitudinal front portion of the garment are separated from the leg elastics disposed in the longitudinal rear portion of the garment by a distance of from about 0.1 to about 50 mm.

3. The absorbent garment as claimed in claim 2, wherein the distance is from about 0.5 to about 15 mm.

4. The absorbent garment as claimed in claim 2, wherein the distance is from about 0.75 to about 5 mm.

5. The absorbent garment as claimed in claim 1, wherein the elastic elements are applied to the garment by stretching them to between about 100% and about 350% of their relaxed length.

6. The absorbent garment as claimed in claim 1, wherein the second elastic element is not adhered to the absorbent core.

7. The absorbent garment as claimed in claim 1, wherein the second elastic element is not adhered to any other element of the absorbent garment.

8. The absorbent garment as claimed in claim 1, wherein the top sheet is at least partially made of a material that is liquid pervious.

9. The absorbent garment as claimed in claim 1, wherein the top sheet is made of a material selected from the group consisting of nonwoven spun-bonded or carded webs of polypropylene, polyethylene, nylon, polyester and blends of these materials.

10. The absorbent garment as claimed in claim 1, wherein the back sheet is at least partially made of a material that is liquid impervious.

11. The absorbent garment as claimed in claim 1, wherein the back sheet is made of a material selected from the group consisting of films of polyethylene, polypropylene, polyester, nylon, polyvinyl chloride and blends of these materials.

12. The absorbent garment as claimed in claim 1, further comprising a fibrous outer liner disposed outside the back sheet.

13. The absorbent garment as claimed in claim 1, further comprising at least two laterally opposing waist containment flaps disposed on the top sheet and extending longitudinally from the front waist region to the rear waist region.

14. The absorbent garment as claimed in claim 1, further comprising releasable fastening mechanisms disposed in the front or rear waist region to enable the front waist region to be releasably attached to the rear waist region.

15. The absorbent garment as claimed in claim 14, wherein the releasable fastening mechanisms are selected from the group consisting of hook and loop type fasteners, pressure sensitive adhesives, snaps, clips, pins, and combinations thereof.

16. The absorbent garment as claimed in claim 1, wherein the leg elastic elements are made from a material selected from the group consisting of natural rubber, lycra, polyurethane, heat shrinkable polymer ribbons, and mixtures and combinations thereof.

17. The absorbent garment as claimed in claim 1, wherein the absorbent core comprises a superabsorbent polymer.

18. A method of forming an absorbent garment comprising:
providing along a machine direction a continuous supply of a top sheet material to a forming station;
providing along a machine direction a continuous supply of back sheet material to the forming station;
providing an absorbent core to the forming station;
providing at least one pair of leg elastic elements to the forming station; and
forming the absorbent garment at the forming station by disposing the absorbent core and the at least one pair of leg elastic elements between the back sheet material and the top sheet material, and cutting the so formed material to form an absorbent garment having the following characteristics:
a) a longitudinal dimension corresponding to the front and back of a wearer, and a lateral dimension corresponding to the left and right side of a wearer, whereby the lateral dimension corresponds substantially to the machine direction of the forming station;
b) laterally extending front and rear waist edges, left and right front side edges, and left and right rear side edges, where at least the front waist edge and left and right front side edges form a front waist region, and at least the rear waist edge and the left and right rear side edges form a rear waist region;
c) a right front arcuate edge and a right rear arcuate edge joining the right front side edge to the right rear side edge, whereby at least part of the arcuate edges form a crotch region that is disposed between the front and rear waist regions, and when the front and rear waist regions are brought together, the left and right, front and rear, arcuate edges define leg openings; and
d) at least a pair of longitudinally opposing leg elastic elements that are disposed adjacent the left and right, front and rear, arcuate edges, and that extend laterally across the crotch region of the garment, whereby the leg elastics are comprised of at least three segments,
whereby providing the at least one pair of longitudinally opposing leg elastic elements comprises supplying at least one elastic element to the longitudinal front half of the garment, and at least one elastic element to the longitudinal rear half of the garment, whereby the elastic elements are supplied in a staggered fashion such that the elastic element supplied to one half of the garment is supplied shortly after the elastic element
supplied to the other half of the garment, supplying the at least one elastic element to the front or rear half of the garment comprises supplying a continuous elastic element in a sinusoidal configuration so that the elastic element includes three different segments:
a) the first elastic element segment being disposed adjacent the front or rear arcuate edge of the garment leading in the machine direction;
b) the second elastic element segment laterally extending across the crotch region of the garment; and
c) the third elastic element segment being disposed adjacent the laterally opposing front or rear arcuate edge of the garment trailing in the machine direction,
whereby the leg elastic elements disposed in the longitudinal front portion of the garment do not contact the leg elastics disposed in the longitudinal rear portion of the garment, the first and third elastic element segments of the longitudinally opposing leg elastics are adhered to at least an element of the garment under tension, and the second elastic element segment is supplied in such a fashion that it is not adhered to another element of the garment under tension, and such that it is under no tensile load after application.

19. The method of claim 18, wherein top sheet material, the back sheet material, and the absorbent core are provided such that the absorbent core is intermittently supplied at a predetermined interval and is disposed between the top sheet material and the back sheet material.

20. The method of claim 18, wherein the leg elastic elements disposed in the longitudinal front portion of the garment are separated from the leg elastics disposed in the longitudinal rear portion of the garment by a distance of from about 0.1 to about 50 mm.

21. The method of claim 20, wherein the distance is from about 0.5 to about 15 mm.

22. The method of claim 20, wherein the distance is from about 0.75 to about 5 mm.

23. The method of claim 18, wherein supplying the continuous elastic element in a sinusoidal configuration comprises supplying the elastic material with a first and second elastic supply mechanism that move back-and-forth in the cross machine direction, whereby the distance the first and second elastic supply mechanisms move in the cross-machine direction is the amplitude, and the distance the first and second elastic supply mechanisms travel in the machine direction during one-half of the sinusoidal application is the frequency.

24. The method of claim 23, wherein the amplitude is from about 30 mm to about 280 mm.

25. The method of claim 24, wherein the amplitude is from about 125 mm to about 175 mm.

26. The method of claim 23, wherein the frequency is from about 50 mm to about 250 mm.

27. The method of claim 26, wherein the frequency is from about 100 mm to about 150 mm.

28. The method of claim 23, wherein the amplitude divided by the frequency is from about 0.1 to about 10.

29. The method of claim 28, wherein the amplitude divided by the frequency is from about 1 to about 3.

30. The method of claim 18, wherein the elastic elements are applied to the garment by stretching them to between about 100% and about 350% of their relaxed length.

31. The method of claim 18, wherein the second elastic element is not adhered to the absorbent core.

32. The method of claim 18, wherein the second elastic element is not adhered to any other element of the absorbent garment.

33. The method of claim 18, wherein the top sheet is at least partially made of a material that is liquid pervious.

34. The method of claim 18, wherein the top sheet is made of a material selected from the group consisting of nonwoven spun-bonded or carded webs of polypropylene, polyethylene, nylon, polyester and blends of these materials.

35. The method of claim 18, wherein the back sheet is at least partially made of a material that is liquid impervious.

36. The method of claim 18, wherein the back sheet is made of a material selected from the group consisting of films of polyethylene, polypropylene, polyester, nylon, polyvinyl chloride and blends of these materials.

37. The method of claim 18, further comprising providing along a machine direction at least two laterally opposing waist containment flaps and disposing the waist containment flaps on the top sheet so that they extend longitudinally from the front waist region to the rear waist region.

38. The method of claim 18, further comprising providing releasable fastening mechanisms after forming the absorbent garment, and disposing the releasable fastening mechanisms on the front or rear waist region to enable the front waist region to be releasably attached to the rear waist region.

39. The method of claim 38, wherein the releasable fastening mechanisms are selected from the group consisting of hook and loop type fasteners, pressure sensitive adhesives, snaps, clips, pins, and combinations thereof.

40. The method of claim 18, wherein the leg elastic elements are made from a material selected from the group consisting of natural rubber, lycra, polyurethane, heat shrinkable polymer ribbons, and mixtures and combinations thereof.

41. The method of claim 18, wherein the absorbent core comprises a superabsorbent polymer.
